(19) United States
(12) Patent Application Publication
(10) Pub. No.: US 2025/0186228 A1
(43) Pub. Date: Jun. 12, 2025

(54) A PASSIVE COMPLIANT WRIST FOR UPPER LIMB PROSTHESIS

(71) Applicant: LIFE AND LIMB PRIVATE LIMITED, Kanpur, Uttar Pradesh (IN)

(72) Inventors: Nishant AGARWAL, Ghaziabad (IN); Prachi KHARB, Chandigarh (IN)

(73) Assignee: LIFE AND LIMB PRIVATE LIMITED, Kanpur, Uttar Pradesh (IN)

(21) Appl. No.: 18/845,403

(22) PCT Filed: Jun. 6, 2022
(86) PCT No.: PCT/IB2022/055243

§ 371 Date: Sep. 9, 2024

(30) Foreign Application Priority Data
Mar. 9, 2022 (IN) 202211012694

(51) Int. Cl. A61F 2/58; A61F 2/54

(52) U.S. Cl. CPC A61F 2/585; A61F 2/586; A61F 2002/543

(57) ABSTRACT

The present disclosure relates to the field of artificial limbs for amputees and specifically to a passive compliant wrist unit 210 for upper limb prosthesis. The passive compliant can comprise a plurality of fingers controlled by a compliant four bar mechanism. The arm is capable of extension/flexion, radial/ulnar deviation on application of a force on the snap joint. The limb is also capable of pronation/supination actions. By application of a force on an arm snap joint, the flexible wrist 216 can be adapted to perform extension/flexion, radial/ulnar deviation.

### TECHNICAL FIELD

The present disclosure relates to the field of artificial limbs for amputees and specifically to a passive compliance wrist unit for upper limb prosthesis.

### BACKGROUND

Background description includes information that may be useful in understanding the present invention. It is not an admission that any of the information provided herein is prior art or relevant to the presently claimed invention, or that any publication specifically or implicitly referenced is prior art.

Available prosthetic wrist units are bulky and the conventional design restricts movements of the wrist thus affecting the Activities of Daily Living [ADLs] that the users can perform with a fully functional wrist. The number of degrees of freedom available with the traditionally available types of artificial limbs are limited. At present, many researchers have tried to include a universal joint, cross-bearings, and tendon driven motions but all of them requires additional requirement of space, power, or mechanical parts. Moreover, the cross section of the available wrists being circular, this makes the overall appearance of the prostheses robotic and the design being bulky at the wrist for the amputees with longer stump length.

Available prosthetic hands have a rotatable wrist unit that limits the user from performing radial/ulnar deviations as well as Extension/Flexion since the joint can only allow supination/pronation of the hand while having circular bearing at the wrist joint.

Chinese patent application CN86206993 discloses a self-adapting general-purpose flexible mechanical hand mechanism, and is a flexible type mechanical hand and a hand mechanism of robot. The utility model is characterized in that the mechanical hand mechanism is an automatic coordinating linkage acting flat surface or a space flexible mechanism, and can grab various of objects or work pieces of different shapes and sizes. The utility model is a ‘hand’ with multifunction, is flexible, fast, stable and safe, and has self-adapting ability and general-purpose property. The utility model has the advantages of simple structure, simple operation, low cost and high loading bearing. The self-adapting general-purpose flexible mechanical hand mechanism can be made into various types and serial flexible mechanical hands and hand mechanisms of robot, using for a plurality of aspects, such as industry, transport, development of ocean, artificial limb, etc.

European Patent EP2500000 discloses a passive artificial limb, comprising: a socket arranged at a first end of the prosthetic limb and exhibiting a shape designed to about a stump of a subject's limb, a terminal part, optionally shaped as a hand or a foot, arranged at a second end of the passive prosthetic limb opposite the first end, a connecting part interposed between the socket and the terminal part. The connecting part comprising at least an adjusting device configured such as to vary at least a moment of inertia of the passive prosthetic limb with respect to at least a reference axis.

There is, therefore, a need to provide a robust, durable prosthetic limb that can perform Extension/Flexion, Radial/Ulnar Deviation, and Supination/Pronation of the hand.

The first two functions, Extension/Flexion, Radial/Ulnar Deviation (A & B) are accommodated in the wrist joint and the function of Supination/Pronation is designed based on the length of the residual limb at the proximal end of the prostheses to address the anatomically similar shape of the wrist.

### OBJECTS OF THE PRESENT DISCLOSURE

Some of the objects of the present disclosure, which at least one embodiment herein satisfies are as listed herein below.

An object of the present disclosure is to provide for Extension and Flexion of the wrist with the application of force from the other hand of the amputee or from external stimulus.

Another object of the present disclosure is to provide Radial and Ulnar Deviations of the hand with the application of force from the other hand of the amputee or from external stimulus.

Another object of the present disclosure is to provide Supination and Pronation of the wrist with the help of other hand of the amputee using snap-clip buttons.

Another object of the present disclosure is to provide a Compliant and water-resistance joint having no mechanical moving parts.

Another object of the present disclosure is to provide a prosthetic limb that allows for user customization of the size of the wrist matching with the anatomical features of the other hand of the amputee based on a 3D scan of the hand.

Another object of the present disclosure is to provide Provision of electronic wiring to route from the palm to the socket for the Electromyography sensors.

### SUMMARY

Aspects of the present disclosure relate to the field of artificial limbs for amputees and specifically to a passive compliance wrist unit for upper limb prosthesis.

The present disclosure relates to the field of artificial limbs for amputees and specifically to a passive compliant wrist unit for upper limb prosthesis. The passive compliant arm comprises a plurality of fingers controlled by a compliant four bar mechanism. The limb is capable of extension/flexion, radial/ulnar deviation on application of a force on the snap joint. The limb is also capable of pronation/supination actions. By application of a force on an arm snap joint, the flexible wrist can be adapted to perform extension/flexion, radial/ulnar deviation.

A passive compliant prosthetic hand is disclosed. The passive compliant prosthetic hand can be comprised of a compliant wrist joint between a wrist and a prosthetic forearm to perform flexion/extension and radial/ulnar deviations. The said prosthetic hand can comprise an arm snap joint that may enable any or a combination of supination and pronation movements.

In an aspect, the passive compliant prosthetic hand may comprise a wrist and a forearm that are adapted to be coupled through any or a combination of male/female connectors installed onto each of the wrist and the forearm.

In an aspect, the passive compliant prosthetic hand may comprise couplings/connectors that may comprise a USB charging means.

In an aspect, the passive compliant prosthetic hand may comprise of male/female connectors that are locked in place by a projection lock.

In an aspect, the passive compliant prosthetic hand may comprise a flexible wrist joint.

In an aspect, the passive compliant prosthetic hand may comprise an arm snap joint. The arm snap joint may be connected with a stump of a person by a prosthetic socket.

In an aspect, the passive compliant prosthetic hand can comprise five digits. The five digits can have a compliant four-bar mechanism that can be actuated by one or more linear servo motors.

In an aspect, the passive compliant prosthetic hand may comprise a control board, a display board and a processor. The hand may comprise any or a combination of a control board, display board and a processor.

In an aspect, the passive compliant prosthetic hand may be force actuated to execute the actions. Once the snap fit joint is locked, the wrist may be enabled to perform any or a combination of a supination or pronation motions.

In an aspect, the passive compliant prosthetic hand may be locked by a locking effect brought about by projection teeth of one or more clips at the joint. The snap-fit joint of the prosthetic forearm may be locked into place by a locking effect between the wrist and the forearm. The locking effect may be by one or more clips having projection teeth, and the snap-fit joint may be unlocked by pressing the clips and rotating the wrist by 45 degrees.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates circular wrist joints that are already known in the art.

FIG. 2 illustrates an exemplary embodiment showing the prosthetic arm consisting of the forearm shells, the wrist units in accordance with the present disclosure.

FIG. 3 illustrates an exploded view of an exemplary embodiment of the prosthetic arm with male and female forearm shells and the male and female wrist units in accordance with the present disclosure.

FIG. 4 illustrates an exemplary embodiment of the female wrist unit of the prosthetic arm in accordance with the present disclosure.

FIG. 5 illustrates an exemplary embodiment of the male wrist unit of the prosthetic arm in accordance with the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure

Exemplary embodiments will now be described more fully hereinafter with reference to the accompanying drawings, in which exemplary embodiments are shown. This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Aspects of the present disclosure relate to the field of artificial limbs for amputees and specifically to a passive compliance wrist unit for upper limb prosthesis.

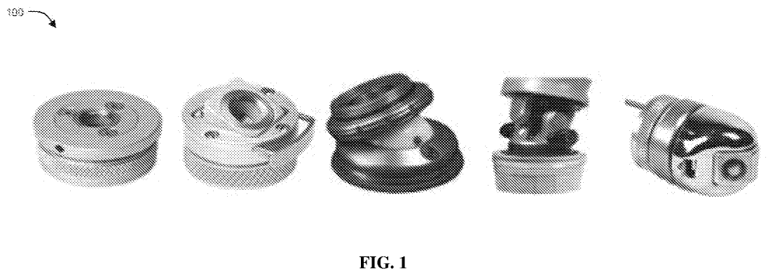

FIG. 1 illustrates circular wrist joints 100 that are already known in the art. A can be seen, all the existing wrist joins are circular in shape and bulky. The present disclosure teaches a passive compliant arm comprising a flexible wrist.

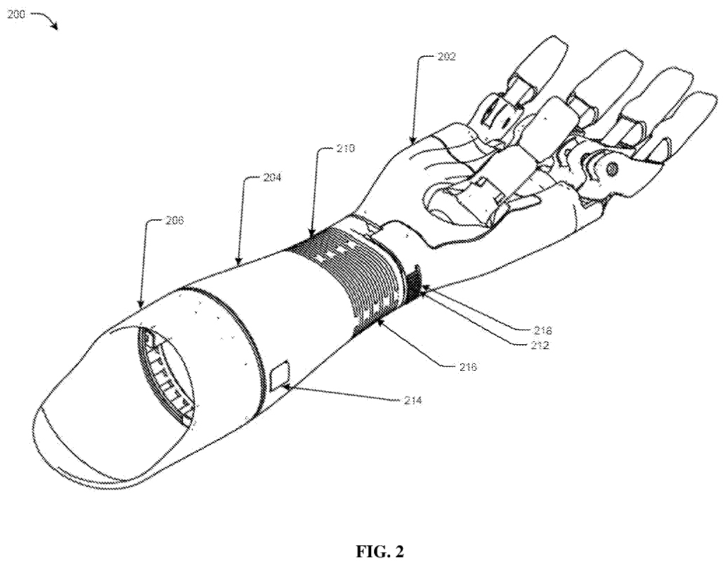

FIG. 2 illustrates an exemplary embodiment showing the prosthetic arm 200 consisting of the forearm shells, the wrist units in accordance with the present disclosure. The prosthetic arm 200 comprises the palm unit 202 adapted with the wrist unit 212 through snap buttons 218. The wrist unit 210 is configured with the forearm shell. The forearm shell may comprise a flexible wrist portion. The male forearm shell 204 may comprise one or more snap buttons 214. The female forearm shell 206 may be configured to receive a male forearm shell 204.

In an aspect of a passive compliant prosthetic arm, the arm may be comprised of a compliant wrist joint. The complaint wrist joint may be comprised of a male wrist unit and a female wrist unit with the female wrist unit being adapted to receive a male wrist unit on a first longitudinal end. The female wrist unit may extend into a flexible wrist on a second longitudinal end of the female wrist unit. The arm may comprise a male forearm shell that longitudinally extends from the flexible wrist and adapted to be configured with a female forearm shell, said female forearm shell adapted to receive the male forearm shell on a first longitudinal end and extending into a prosthetic socket on a second longitudinal end.

In an aspect, the passive compliant prosthetic comprises the flexible wrist of the male forearm shell being capable of extension, flexion, radial deviation and ulnar deviation.

In another aspect the male forearm shell may comprise one or more snap buttons. The snap buttons may be adapted to enable any or a combination of supination and pronation movements of the arm on application of a force on the snap button of the male forearm shell.

In another aspect, the male wrist unit may be adapted to receive a palm unit with the palm unit having a plurality of digits.

In yet another aspect, the male wrist unit may comprise a lock head to enable locking with the female wrist unit, one or more snap buttons to enable application of a force on the wrist joint and a thumb pillar to enable fitment of the male wrist unit with the female wrist unit.

In yet another aspect, the prosthetic arm is connected with a stump of a person by the prosthetic socket.

In yet another aspect, the palm unit may comprise five digits that are moveable by a compliant four-bar mechanism actuated by one or more linear servo motors.

In yet another aspect, the female wrist unit may comprise snap-buttons that enable any or a combination of supination or pronation motions of the prosthetic arm on application of a force on the female wrist unit snap buttons.

In an aspect, the male wrist unit may be provided with a 4-pin spring connect to receive a programmable circuit board configured with the female wrist unit.

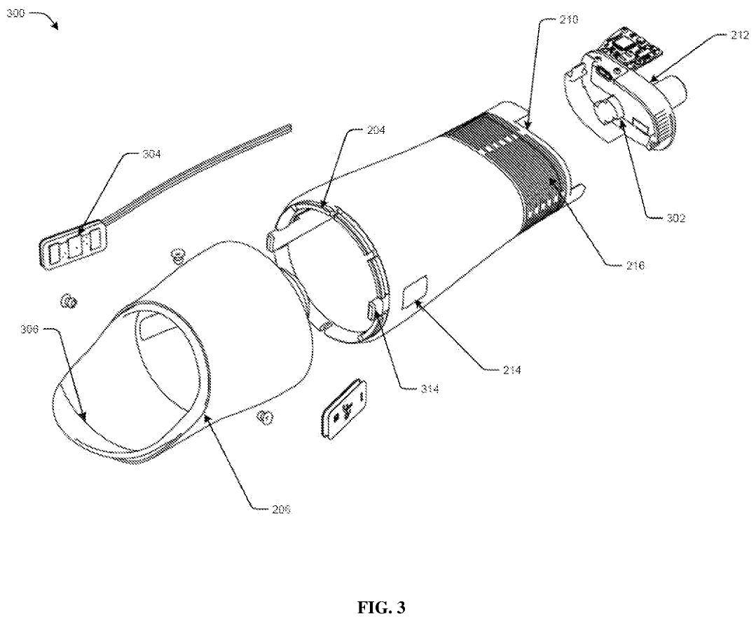

FIG. 3 illustrates an exploded view of an exemplary embodiment of the prosthetic arm 300 with male and female forearm shells and the male and female wrist units in accordance with the present disclosure. The male wrist unit 212 may comprise a locking head 302 that engages with the female wrist unit 210. The female wrist unit 210 may comprise seats/clips to enable engaging and disengaging between the male wrist unit 212 and the female wrist unit 210. The female forearm shell 206 may be connected to snap fit phalanges 314 to receive the male forearm shell 204.

In an aspect, the compliant joint of the prosthetic forearm is locked into place by a locking effect between the male wrist unit and the female wrist unit, said locking effect being by one or more clips having projection teeth, and the compliant joint is unlocked by pressing the clips and rotating the wrist by 45 degrees.

In an embodiment, the passive compliant wrist may comprise a flexible wrist 216 connected to a prosthetic forearm. The flexible wrist 216 may be connected to a connecting arm through a snap joint. The connecting arm may be adapted to connect with a stump of a person using a prosthetic socket 306. The flexible wrist 216 may comprise about five digits that may be controlled using a compliant 4 bar mechanism. Each of the digits may be provided with a plurality of sensors. In the given embodiment, an FSR sensor is shown, that may be connected to a control board and a display board to display the readings. Multiple control boards may be provided to control and monitor different aspects through different sensors. Reference shows a palm side sectional view of the wrist at the flexible joint. Reference shows a side view of the arm side of the joint. Connector boards and may be adapted to mesh with each other to form a stable grip. A USB charger port or a data retriever port may be provided to ease the reading of data collected by the sensors on the digits. In an embodiment, at each of the digits, a PIP Joint angle range from 170 (max. open) to 80 (min. closed) degrees. In an embodiment, at each of the digits, MCP Joint angle measured from the plane of the palm, 175 (max. open) to 100 (min. closed) degrees. A plurality of electromyography sensors (EMG) may be provided in the digits.

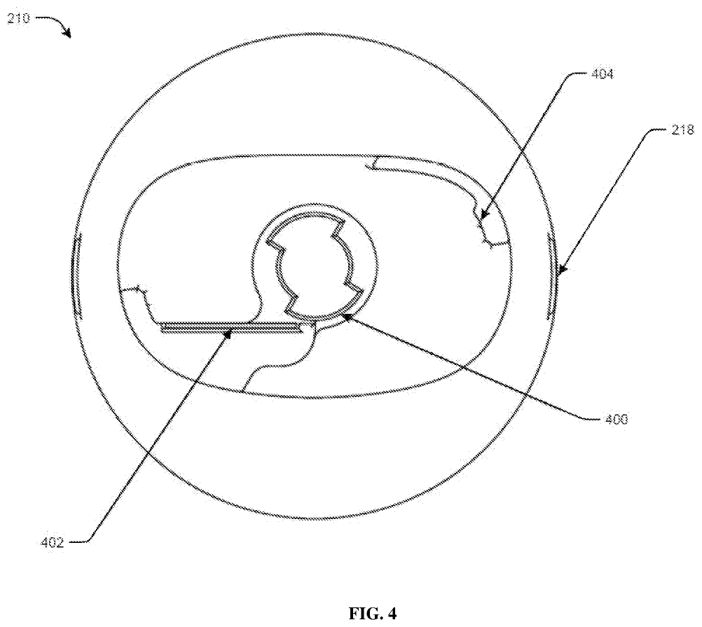

FIG. 4 illustrates an exemplary embodiment of the female wrist unit 210 of the prosthetic arm 300 in accordance with the present disclosure. The female wrist unit 210 may comprise a lock head 400 adapted to receive and lock the male wrist unit 212. A plurality of snap buttons 218 may be configured to enable one of a supination-prionation movements of the arm.

In an embodiment, the passive compliant prosthetic hand may comprise a wrist and a forearm that are adapted to be coupled through any or a combination of male/female connectors installed onto each of the wrist and the forearm.

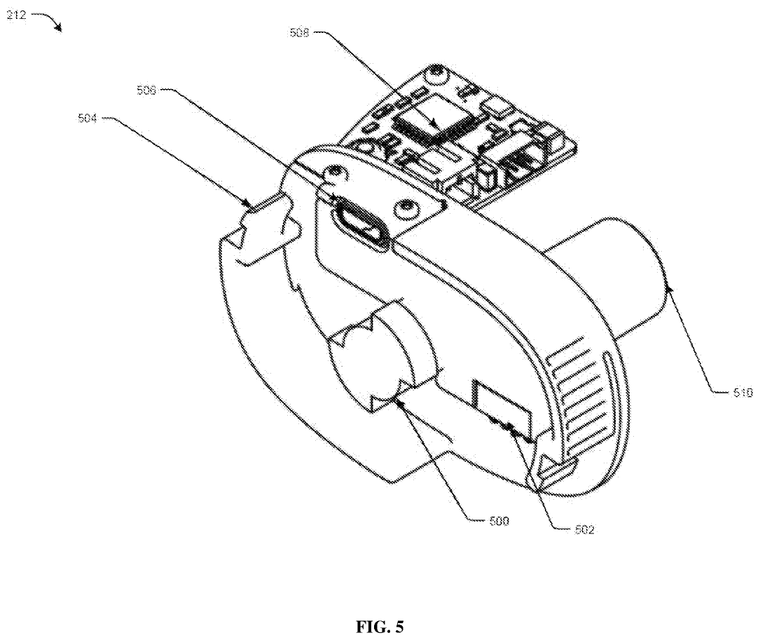

FIG. 5 illustrates an exemplary embodiment of the male wrist unit 212 of the prosthetic arm 300 in accordance with the present disclosure. The male wrist unit 212 may comprise a thumb pillar 510 to enable proper fitment of the male unit 212 with the female unit 210. In an embodiment, the passive compliant prosthetic hand may comprise couplings/connectors that may comprise a USB charging means.

In an embodiment, the passive compliant prosthetic hand may comprise of male/female connectors that are locked in place by a projection lock.

In an embodiment, the passive compliant prosthetic hand may comprise a flexible wrist joint.

In an embodiment, the passive compliant prosthetic hand may comprise an arm snap joint. The arm snap joint may be connected with a stump of a person by a prosthetic socket.

In an embodiment, the passive compliant prosthetic hand can comprise five digits. The five digits can have a compliant four-bar mechanism that can be actuated by one or more linear servo motors.

In an embodiment, the passive compliant prosthetic hand may comprise a control board 508, a display board and a processor. The hand may comprise any or a combination of a control board 508, display board and a processor.

In an embodiment, the passive compliant prosthetic hand may be force actuated to execute the actions. Once the snap fit joint is locked, the wrist may be enabled to perform any or a combination of a supination or pronation motions.

In an embodiment, the passive compliant prosthetic hand may be locked by a locking effect brought about by projection teeth of one or more clips at the joint. The snap-fit joint of the prosthetic forearm may be locked into place by a locking effect between the wrist and the forearm. The locking effect may be by one or more clips having projection teeth, and the snap-fit joint may be unlocked by pressing the clips and rotating the wrist by 45 degrees.

In an embodiment, a flexible wrist 216 is connected to a prosthetic forearm and a connecting arm. The flexible wrist 216 may be connected to the forearm via a snap fit joint. When a force is applied on the snap fit joint, for instance, the snap fit joint is pressed by a hand, the snap fit joint makes the prosthetic arm 300 free to rotate along a longitudinal axis passing through the entire length of the arm 300. These motions are referred to as supination-pronation motions of an arm. The snap-fit joint may have a locking feature. The snap-fit joint which when pressed by the other hand, makes the arm free to rotate in either direction. The prosthetic forearm and the connecting arm are assembled only once and can only be detached using special tools to avoid accidental dismantling of the hand.

In an aspect, a projection lock may replace the snap-fit joint, The projection lock may have male and female parts that lock-in when the two parts are brought together. The reference numeral indicates the direction of motion allowed when the joint is locked.

In an exemplary embodiment, the prosthetic arm 300, may include a flexible wrist 216, a prosthetic forearm and a connecting arm. The flexible wrist 216 is capable of rotating about a longitudinal axis passing through the length of the prosthetic arm 300 when the projection lock is locked.

In an embodiment, the flexible wrist 216 may have a projection that may have quarter teeth to adequately fit into the prosthetic fore-arm. When rotated by 45 degrees, the lock releases and is free to translate to remove the flexible wrist 216 from the prosthetic forearm. This fit may happen with two clips of the wrist when the two clips are pressed and rotated simultaneously.

The Extension/Flexion, Radial/Ulnar Deviation motions of a passive compliant wrist for upper limb prosthesis may be actuated on application of a force. The flexible wrist 216 can perform RADIAL/ULNAR Deviations and EXTENSION/FLEXION using the external stimulus or my using the other hand of the amputee. The material used for making this compliant wrist may be NYLON-12 which is fatigue resistant and provides performance and design advantages.

In an embodiment, the actuation of the one of the radial/ulnar deviations, the extension/flexions may be configured to be automatically generated on the receipt of one or more signals from the sensors in the digits. The extension/flexion may be at an angle of 50 degrees from the horizontal axis. The radial/ulnar deviation may be at an angle of 40 degrees from the horizontal axis.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Present Disclosure

Some of the advantages of the present disclosure, which at least one embodiment herein satisfies are as listed herein below.

The present invention enables Extension and Flexion of the wrist with the application of force from the other hand of the amputee or from external stimulus.

The present invention enables Radial and Ulnar Deviations of the hand with the application of force from the other hand of the amputee or from external stimulus.

The present invention enables Supination and Pronation of the wrist with the help of other hand of the amputee using snap-clip buttons.

The present invention enables a Compliant and water-resistance joint having no mechanical moving parts.

The present invention provides a prosthetic limb that allows for user customization of the size of the wrist matching with the anatomical features of the other hand of the amputee based on a 3D scan of the hand.

The present invention enables the provision of electronic wiring to route from the palm to the socket for the Electromyography sensors.

We claim:

1. A passive compliant prosthetic arm, said arm comprising:
a compliant wrist joint comprised of a male wrist unit (212) and a female wrist unit (210), said female wrist unit (210) adapted to receive a male wrist unit (212) on a first longitudinal end and extending into a flexible wrist (216) on a second longitudinal end;
a male forearm shell (204) longitudinally extending from the flexible wrist (216) and adapted to be configured with a female forearm shell (206);
said female forearm shell (206) adapted to receive the male forearm shell (204) on a first longitudinal end and extending into a prosthetic socket (306) on a second longitudinal end.

2. The passive compliant prosthetic arm as claimed in claim 1, wherein the flexible wrist (216) of the male forearm shell (204) enables extension, flexion, radial deviation and ulnar deviation.

3. The passive compliant prosthetic arm as claimed in claim 1, wherein male forearm shell (204) comprises one or more snap buttons (214), said snap buttons (214) adapted to enable any or a combination of supination and pronation movements of the arm on application of a force on the snap button (214) of the male forearm shell (204).

4. The passive compliant prosthetic arm as claimed in claim 1, wherein the male wrist unit (212) is adapted to receive a palm unit (202), said palm unit (202) having a plurality of digits.

5. The passive compliant prosthetic arm as claimed in claim 1, wherein the male wrist unit (212) comprises a lock head (302) to enable locking with the female wrist unit (210), one or more snap buttons (214) to enable application of a force on the wrist joint and a thumb pillar to enable fitment of the male wrist unit (212) with the female wrist unit (210).

6. The passive compliant prosthetic arm as claimed in claim 1, wherein the arm is connected with a stump of a person by the prosthetic socket.

7. The passive compliant prosthetic arm as claimed in claim 4, wherein said palm unit (202) comprises five digits moveable by a compliant four-bar mechanism actuated by one or more linear servo motors.

8. The passive compliant prosthetic arm as claimed in claim 1, wherein the female wrist unit (210) comprises snap-buttons (214) that enable any or a combination of supination or pronation motions of the prosthetic arm on application of a force on the female wrist unit snap buttons.

9. The passive compliant prosthetic hand as claimed in claim 1, wherein the male wrist unit (212) is provided with a 4-pin spring connect (502) to receive a programmable circuit board configured with the female wrist unit (210).

10. The passive compliant prosthetic hand as claimed in claim 1, wherein the compliant joint of the prosthetic forearm is locked into place by a locking effect between the male wrist unit (212) and the female wrist unit (210), said locking effect being by one or more clips having projection teeth, and the compliant joint is unlocked by pressing the clips and rotating the wrist by 45 degrees.
